(19) United States
(12) Patent Application Publication
(10) Pub. No.: US 2001/0031739 A1
(43) Pub. Date: Oct. 18, 2001

(54) Method and kit for quantitating genomic DNA damage and repair capicity

(76) Inventor: Akintade Oyedele Dare, Ft. Washington, MD (US)

Correspondence Address: Lawrence Harbin, McIntyre Harbin & King, One Massachusetts Avenue, N.W., Suite 330, Washington, DC, 20001, US

(21) Appl. No.: 09/741,426

(22) Filed: Dec. 21, 2000

Related U.S. Application Data
(60) Provisional application No. 60/171,309, filed on Dec. 21, 1999.

(51) Int. Cl. C12Q 1/68; A61K 31/70; C12M 1/34; C12M 3/00

(52) U.S. Cl. 514/44; 435/6; 435/287.2

(57) ABSTRACT

A microtiter plate-based assay method requiring no radioactivity and that enables automated assays of DNA oxidative stress. A disclosed method, directly and completely on the microtiter plate, comprises binding DNA to microtiter plate, reacting abasic (AP) sites of DNA with a chemical reagent (aldehyde reactive probe (ARP)), and performing a biotin avidin horseradish peroxidase technique to tag ARP with a biotin residue for subsequent colorimetric analysis. Reacting may be performed on DNA cells in culture. Binding may use Reactibind, which is an Aldehyde-Reactive-Probe. Colorimetric analysis may be performed using an avidin-biotin conjugated mechanism to quantify the tagged abasic (AP) sites in DNA. Also disclosed is a procedure to assay modified bases in genomic DNA by enzymatic digestion using an N-glycosylase enzyme such as endonuclease-III, 8-oxoguanine glycosylase [yOOG1], human 8-oxoguanine glycosylase [hOGG1], and/or an FPG protein to indicate repair capacity. The invention enables automation of DNA assays for large population studies, as well as portable hand-held devices for quantitating DNA damage and repair capacity.

### CROSS-REFERENCE TO RELATED APPLICATION

This invention claims the benefit of U.S. Provisional Application Ser. No. 60/171,309 filed Dec. 21, 1999, in the name of the same inventor hereof, and entitled Quantitative Assay Method for Detecting Abasic Sites and Analyzing DNA Repair in Genomic DNA, which is incorporated herein by reference.

### BACKGROUND

This invention relates to a DNA quantitative assay method and a kit to assay genomic DNA, but more specifically, to a rapid and sensitive DNA assaying method using a calorimetric procedure to visually detect abasic sites in DNA.

Free radicals, or reactive oxygen species (ROS), are generated in vivo by a variety of mechanisms that include oxidative stress, redox cycling, and ionizing radiation. Several cellular processes endogenously generate superoxide anions which, in turn, generate hydroxyl radicals in the nuclei of cells. This causes the formation of abasic (AP) sites resulting from removal of purine or pyrimidine bases and site specific damage to DNA. Depending on the mechanism of generating free radicals, free radical induced oxidative DNA base modifications include 8-hydroxy-guanine, thymine glycol, formamido residues, dihydrothymine, 5-hydroxymethyl uracil, and 5-hydroxy-5-methylhydantoin. Base excision repair pathways (BER) of the cell repair these base modifications. In its initial step, the BER pathway results in the formation of additional abasic (AP) sites. Deficiency in the cell's DNA base excision repair pathway (BER) and increased oxidative stress contribute to increased background levels of AP sites in cells and tissues.

Interestingly, recent studies suggest that certain human diseases create increased background level of oxidative DNA damage during their pathogenesis. These include Alzheimer's disease (Mecocci et al., 1994, Ann Neurolog. 36:747-651; Prashad et al., 1996, Proc. Natl. Acad. Sci. USA 93:5146-50), amyotrophic lateral sclerosis, Parkinson's disease, cataract formation, aging process, radiation exposure (Wilson et al., 1992, Cancer Res. 48:2156-2162) ischemic damage and stroke (1996, NIH Guide 25), metal toxicity carmichael et al., 1995, Mutat. Research 326:235-43), breast cancer (Djuric et la., 1996, Cancer 77:691-6), carcinogenesis (Ames et al., 1995, Proc. Natl. Acad. Sci. USA 92:5258-65). Molecular mechanisms, including the pathogenesis of oxidative DNA damage and alteration of a cell's ability to repair damaged DNA, may lead to the development of genomic instability. Genomic instability is believed to occur in an early step in the process of carcinogenesis. In addition, cells and human tissues are being screened for specific DNA damage in order to correlate the action of DNA damaging agents with human diseases and to verify the contribution of DNA damaging agents in specific genetic states to manifestation of human diseases.

Prior methods for quantifying abasic (AP) sites in DNA have been reported but only some allow discrimination of sparse or a low number, e.g., concentration, of AP sites in DNA. Some of these methods require the use of radioactive materials which endangers the health of the operators and contaminates the environment. Others not requiring radioactive material are either extremely time consuming, technique-sensitive, cumbersome and not cost-efficient. A previous method addressing these challenges used Aldehyde Reactive Probe (ARP) to tag biotin to aldehyde groups of AP sites in DNA (Kubo et al, 1992). The ELISA-like method (enzyme-linked immunosorbent assay) it use, however, was limited in its sensitivity. In addition, the method used the laborious step of ethanol precipitation/centrifugation to remove excess ARP that limited the effectiveness of the assay with small DNA samples.

Accordingly, there is a need for sensitive assays for specific lesions resulting from oxidative DNA damage. It is known to those skilled in the art that exposure of an individual to ionizing radiation or certain chemotherapeutic agents or toxic agents can result in oxidative DNA damage. Thus, there is a need for methods, kits, and apparatuses according to the present invention which are clinically useful to assess oxidative stress brought about by, for example, exposure to toxic agents, chemotherapy or radiation therapy. In addition, for individuals at high risk of a pathological condition associated with oxidative DNA damage, it may be useful to monitor levels of abasic site and specific DNA base modifications resulting from oxidative DNA damage prior to clinical onset of the condition.

Several methods for quantifying AP sites have been reported, and some allow discrimination of low AP sites in DNA with the use of radioactive methods. These include methods disclosed by U.S. Pat. No. 5,906,918 to Box, et al. Other methods, such as those disclosed by U.S. Pat. No. 6,048,969 to Hoffman et al. and U.S. Pat. No. 4,794,074 to Harris, while not requiring radioactive treatment are time consuming, technique-sensitive, and/or cumbersome. In addition, with the exception of thymine glycol which can be measured using an immunoassay (Hubbard-Smith et al., 1992, Radiat. Res. 130:160-5; U.S. Pat. No. 5,552,285) or by mass spectrometric analyses (Markey et al., 1993, Ann. NY Acad. Sci. 679:352-7), quantitation of each specific DNA base modification has been hindered by the lack of sensitivity of existing assays to detect biologically significant levels of each category or type of base modification amongst the variety of base modifications that may be present.

The present invention addresses the above and other challenges in the field of genomic DNA analysis in providing rapid and sensitive measurement of AP sites in DNA while directly bound to a microtiter plate thereby obviating any need for ethanol precipitation or ultrafiltration/centrifugation or other processes. In addition, the present invention enable miniaturization of the assay kits and portable, hand-held DNA analysis devices; does not employ environmental unfriendly radioactive methods and materials; permits the quantitative detection of specific base damage in a spectrum of base modifications using substrate-specific enzymes; is suitable for clinical application and population and epidemilogical studies; and above all can be automated for quantitative detection of abasic sites in a large number or concentration of DNA samples.

### SUMMARY OF THE INVENTION

According to a first embodiment of the invention, there is provided a simplified method of assaying DNA comprising the steps of (i) binding to an analysis plate, such as a polystyrene microtiter plate, both sample DNA under examination and control DNA having known abasic sites, (ii) treating the respective samples in parallel with a labeling reagent, such as aldehyde reactive probe (ARP) (N′-aminooxymethylcarbonylhydrazino-D-biotin) reagent, and (iii) using an ELISA-like method to detect abasic sites tagged with biotin after the reacting step wherein the ELISA-like method includes an avidin- biotin-complex conjugated with horseradish peroxidase or alkali phosphatase. In the ARP assay, for example, absorbance was measured at 650 nm wavelength and signals were expressed as the change in absorbance after subtracting background reading of the control DNA. Ordering of the steps may be altered. For example, the sample and control DNA may be tagged or labeled separately with a biotin residue of the ARP reagent, and then bound to the analysis plate for comparison. That is to say, for example, the method may be practiced by reacting ARP with AP sites of DNA of cells “in culture” before the binding step since ARP is selectively permeable to cell membranes.

Control DNA preferably is a heat/acid buffer depurination of calf thymus (Lindahl & Nyberg, 1972, Kubo, et al., 1992), which is used as a control against which the sample DNA is compared. Treating is preferably but need not be performed in parallel so that the sample and control DNA specimen(s) are each subjected to the same or similar environmental and/or process conditions so as to remove any such variables from the respective treated samples when interpreting the results of their comparison(s). Binding of untreated (before ARP-reaction) or treated (biotinylated) DNA to the analysis plate may be accomplished using a Reacti-bind solution. Comparing or quantifying may be accomplished by performing a standard ELISA-like, colorimetric analysis, e.g., detecting optical density, after performing an avidin biotin complex conjugated with horseradish peroxidase or alkali phosphatase to indicate relative optical densities of the sample DNA and the standard DNA specimen(s).

In accordance with another aspect of the invention, the method additionally includes quantitatively assaying DNA damage, i.e., determining the number of abasic sites, by comparing sample DNA with multiple control DNA specimens. In yet another aspect of the invention, the method includes determining repair capacity of sample DNA by comparing relative enzyme activity levels of the sample and control DNA. In a preferred method, repair capacity is determined by observing the relative response of the sample and control DNA to an enzyme selected from the group of Endonuclease III, 8-oxoguanine glycosylase [yOGG1], human 8-oxoguanine glycosylase [hOGG1].

According to another embodiment of the invention, there is provided a method of (i) binding DNA to an analysis plate, such as a microtiter plate, using Reacti-bind, (ii) removing the excess Reacti-bind and unbound DNA using a detergent so as not to remove the bound DNA, (iii) reacting the bound DNA with an excess amount of aldehyde reactive probe (ARP), (iv) removing the excess and unreacted ARP from the analysis plate, (v) labeling/tagging the attached ARP using a biotinylated chemical agent, and (vi) performing a colorimetric analysis to quantitatively assess the sample DNA relative to the control DNA attached to the plate.

The method may further include binding to the analysis plate a relatively high percentage of DNA contained in a solution of relatively low concentration being in the range of 1.0 to 10.0 nano grams per milliliter, and by using Reactibind to attach the sample and control DNA to a microtiter plate. This achieves a relatively high lesion sensitivity in the order of less than one abasic site per 100,000 bp. Such low concentrations of sample DNA enable assaying of DNA samples taken from, for example, the buccal epithelium of a subject, rather than invasively obtaining large quantities of blood, tissue, or cells; or culturing samples over a long period of time, as required by prior procedures.

According to yet another embodiment of the invention, there is provided a simplified kit for assaying sample DNA comprising an analysis plate, such as a microtiter plate; a number of control DNA specimens, each having a known number or concentration of abasic site; a surface treatment solution to enhance attachment of the DNA to the analysis plate; a detergent for removing excess material from the plate; an aldehyde reactive probe (ARP) to label or tag abasic sites while attached to the plate; a calorimetric test kit such as a kit for performing an avidin-biotin horseradash peroxidase technique; and instructions to carry out one of the methods described herein. Binding to the analysis plate may be accomplished using a Reacti-bind solution and the comparison may be accomplished by performing a standard calorimetric analysis, such as by using an avidin-biotin-horseradish peroxidase technique, with respect to the sample DNA and the standard DNA specimen(s).

In yet a further aspect of the invention, DNA repair capacity is assayed by subjecting the sample and/or control DNA to a substrate-specific repair enzyme, tagging the product of the enzyme reaction, binding the DNA to an analysis plate, and determining the resulting number of abasic sites remaining on the analysis plate after the enzyme reaction whereby to assay the ability of the cell to undergo DNA repair. Preferably, the DNA is subjected to a DNA glycosylase selected from the group of endonuclease III, 8-oxoguanine glycosylase [yOOG1], human 8-oxoguanine glycosylase [hOGG1], FPG protein, 8-oxoguanine, alkA protein, or other DNA glycosylase, broad or narrow spectrum.

Various other assays may be practiced using the analysis methods hereof. The above are only exemplary.

In accordance with yet a further aspect of the invention, an apparatus that automates DNA assaying for large population studies includes an analysis plate to which sample and control DNA is bound, a number of processing stations that perform process activities with respect to the analysis plate, and a controller that effects movement of the analysis plate to the processing stations and the operations of the processing stations. Operations performed by the processing stations includes at least one of receiving and dispensing sample and control DNA; reacting a surface treatment agent such as Reacti-bind with the samples to bind them to the plate; washing the plate so as to remove excess surface treatment agent; reacting the bound DNA with an aldehyde reactive probe (ARP) so as to tag or label the DNA bound to the plate; biotinilating the ARP-tagged DNA; washing excess un-reacted ARP from the plate without removal of bound DNA; and performing a calorimetric technique such as an avidin-biotin-horseradash peroxidase technique.

According to another aspect of the invention, the method comprises analyzing sample and control DNA by treating the sample and control in parallel completely on a microtiter plate whereby to provide a simplified, direct assay procedure suitable for automation using process control software.

The invention thus advantageously provides a way to directly estimate in a very sensitive manner and within a few hours the level of oxidative stress/injury in sample DNA in the shortest time possible and in a cost-effective manner, and without using radioactive-emitting substances or methods.

### BRIEF DESCRIPTION OF THE DRAWINGS

FIG. 1 is a graph depicting a linear relationship between an amount of DNA in nanograms bound to the analysis plate, e.g., well-bottoms of a microtiter plate, using Reacti-bind as a surface treatment agent and a concentration in micrograms/milliliter of control DNA (e.g., calf thymus) in the solution applied to the plate.

FIG. 2 is a graph illustrating a linear relationship between an ARP signal indicative of optical density (OD) at 650 nanometer wavelength and a number (zero to five) of AP sites per 10,000 base pairs (bp) for control DNA concentrations of 2.5, 5.0, and 10 micrograms per milliliter.

FIG. 3 is a graph illustrating a linear relationship between an ARP signal indicative of optical density (OD) at 650 nanometer wavelength and the number (zero to one) of AP sites per 10,000 base pairs (bp) for control DNA concentrations of 5.0 and 10 micrograms per milliliter.

FIG. 4 is a graph indicating the number of AP sites generated over a three to nine day incubation period resulting from spontaneous physiological depurination of calf thymus DNA at 4° Celsius and 37° Celsius.

FIG. 5 illustrates an exemplary apparatus for automating assaying of DNA utilizing methods disclosed herein.

### DESCRIPTION OF ILLUSTRATIVE EMBODIMENTS

Generally, DNA that is immobilized to microtiter dishes is assayed using biotin-labeled aldehyde reactive probes treated with avidin-horseradash peroxidase (HRP). AP sites are quantitated by a chromogenic reaction and the method can be computer automated to screen a large number of samples for oxidative damage. The number of AP sites is quantified by measuring HRP chromogenic substance by an ELISA method.

Alternatively, the sample and control DNA may be tagged or labeled separately with a biotin residue of the ARP reagent, and then bound to the analysis plate for comparison. That is to say, for example, a method of the invention may be practiced by reacting ARP with AP sites of DNA of cells in culture before binding the DNA to the analysis plate since ARP is selectively permeable to cell membranes. Once sample DNA and control DNA are tagged while in culture, they are then extracted, isolated, purified, and bound to the plate for further analysis in accordance with the embodiments described herein. This “in culture” ARP reaction provides even greater sensitivity in detecting abasic sites because background noise is completely removed before other steps of the methods even begin.

The assay method described herein provides an accurate, rapid and cost-effective way to count abasic (AP) sites and DNA (deoxyibrose nucleate acid) base modifications in genomic DNA of cells and tissues. Measurements are performed directly, rather than indirectly, and are performed completely on an analysis plate, such as a commercially available microtiter plate, without the need to remove and/or transport samples to other laboratory facilities. Once DNA samples are purified, an assay may be completed within a few hours using methods and apparatuses of the present invention. Apart from the description contained herein, certain aspects of the invention claimed hereby may be found in recent publications predicated on research of the inventor hereof, including Dojindo Newsletter Vol. 2, entitled Oxidative Stress, DNA Damage and Human Diseases published at www.dojindo.com/newsletter/review_vol2.html in the year 2000 by Dojindo Molecular Technologies of Gaithersburg, Md., and Technical Manual: DNA Damage Quantification Kit—AP Site Counting, Dojindo Product Code AK02-12, also found at www.dojindo.com, each of which are incorporated herein by reference.

As a first example, the method comprises isolating and purifying genomic DNA from cells and tissues using conventional methods. In the assay, sample DNA and control DNA are examined together. A DNA coating agent is then used to bind the DNA to a microtiter plate. Aldehyde groups of abasic sites in the bound DNA are subsequently labeled or tagged while bound to the plate using an excess amount of a biotinylated aldehyde reactive chemical reagent. In order to prevent contributory non-specific abasic sites from reacting with excess reagent, the excess reagent was removed without detaching the previously bound DNA. The abasic (AP) sites contained in the tagged, bound DNA of the sample and control specimens were then quantitatively detected chromatographically by an avidin-biotin conjugate method where the DNA sample under examination was compared calorimetrically with standard control specimens of known (i.e., control specimens) DNA base modifications.

Measurements of specific DNA base modifications are carried out at the dimer level. This may be done by digesting with an enzyme the damaged DNA under examination using a substrate specific N-glycosylase enzyme, such as Endonuclease III, formamidopyimidine N-glycosylase (fpg), 8-oxoguanine glycosylase [yOOG1], human 8-oxoguanine glycosylase [hOGG1], and/or an FPG protein. The digestion process also produces a number of abasic sites in DNA. The bound aldehyde group at the abasic site in the DNA thus created was specifically labeled/tagged by an excess of a biotinylated aldehyde reactive chemical reagent. In order to prevent contributory non-specific sites from reacting with excess reagent, excess reagent was removed without detaching the DNA that were bound to the microtiter plate. A chromatographic method incorporating or by means of an avidin-biotin conjugate or similar method using the microtiter plate was then used to quantitatively determine the abasic (AP) sites contained in the bound, tagged DNA.

Since certain DNA base modifications prevent or significantly inhibit hydrolysis of the neighboring phosphodiester bond by an endonuclease, the results of which results in hydrolization of single-stranded DNA, the sample of DNA to be assayed for the presence of DNA base modifications is digested with the endonuclease; and then with a phosphatase which dephosphorylates terminal phosphate groups. An internal DNA standard, similar to the samples, with specific inclusion of base modification was similarly digested and the resulting abasic sites determined as described for accurate and sensitive quantitation. The quantity of abasic sites determined was directly related to the quantity of specific N-gylcosylase sites (base modified substrate) in the damaged DNA.

To produce the control DNA against which the sample DNA is compared, double stranded calf thymus was obtained from Signal Chemical Company and specific numbers of abasic sites were selectively produced, as known in the art. Prior to heat/acid-buffer treatment, the DNA was treated with 5 mM of methoxyamine and then removed by ethanol precipitation. The DNA was then suspended in sodium phosphate buffer, pH 7. The methoxyamine-treated and methoxyarnine non-treated DNA (100 g/mL) were then dialyzed separately in 10 mM NaH₂PO₄, 100 mM NaCl, and 10 mM sodium citrate at pH 5.0 (AP-buffer). The dialyzed control DNA was heated at 70° C. for fifty minutes and the reaction was stopped by chilling rapidly on ice to create 5 AP sites/10,000 bp. Each control DNA specimen was dialyzed back to pH 7.5 in PBS buffer (137 mM NaCl, 2.7 mM KCl, 4.3 mM NaH₂PO 7H20, and 1.4 mM KH₂PO₄. This was diluted with control DNA to produce varied concentrations of, for example, 1, 2, 3, and 4 AP sites/10,000 bp. Desired control DNA concentrations may also be obtained by diluting with PBS buffer. To create low-level AP sites in methoxyamine treated DNA, 1 AP site DNA was diluted with control DNA to produce 0.1, 0.2, 0.4, 0.8 AP sites/10,000 bp. Similar, low-level AP site in non-treated DNA was created by diluting 1 AP site DNA specimen with control DNA to produce 0.2, 0.4, 0.8, and 1.6 AP sites/10,000 bp.

The methods described herein may be provided in an assay kit useful to detect and quantify abasic (AP) site in a genomic DNA sample isolated from cells and tissues. When the method includes N-glycosylase (endonuclease) enzyme-coupling, certain other DNA base modifications can be detected. These include the detection and quantitation of 5-hydrox-5-methylhydantoin, 5-hydroxymethyluracil, and 8-hydroxyguanine.

The method provides greater rapidity, sensitivity, simplicity and cost-effectiveness than prior methods, and is more accurate in detecting and quantifying abasic sites in human genomic DNA to indicate genomic instability. The invention also enables automated analysis of oxidative stress in a large number samples for clinical application and population studies.

Importantly, the invention does not require radioactive procedures to detect abasic (AP) sites in DNA. Because assaying occurs directly and completely on a microtiter plate, it is feasible for complete automation and full integration with an information processing and control systems.

### EXAMPLE 1

A first procedure includes immobilizing and binding the sample and/or control DNA to respective wells of a polymer-based microtiter plate, such as a 96-well U-bottom microtiter Costar #3791 plate, commercially available from Costar Corporation of Cambridge, Mass. The surface or plate to which the DNA is to be bound may include surface treatment to enhance binding. Providing a nitrocellulose membrane is one such surface treatment.

To enhance immobilization and attachment of the DNA to the microtiter plate, 200 μL solutions of sample DNA concentrations of 2.5, 5, or 10 μg/mL were respectively prepared. This provided a basis to analyze the DNA sample for three different concentrations of the sample DNA under examination. In each case, the 200 μl solution was mixed with 300 μL of Reacti-bind DNA coating solution, which is commercially available from Pierce Chemical Corporation of Rockford, Ill. Next, 100 μL of the mixture was added to each well of the microtiter plate.

Next, the microtiter plate with the DNA bound thereto was incubated at room temperature for eight to sixteen hours. Unbound DNA was removed by washing the plate three to five times with a 0.1% Tween-PBS buffer solution (phosphate buffered solution containing 0.1% Tween 20, which is a commercially available biological detergent). Washing is carried out to an extent so as not to remove the DNA bound to the microtiter plate. This method of binding DNA obviates problems associated with using ultraviolet light to enhance DNA immobilization, which is not very efficient.

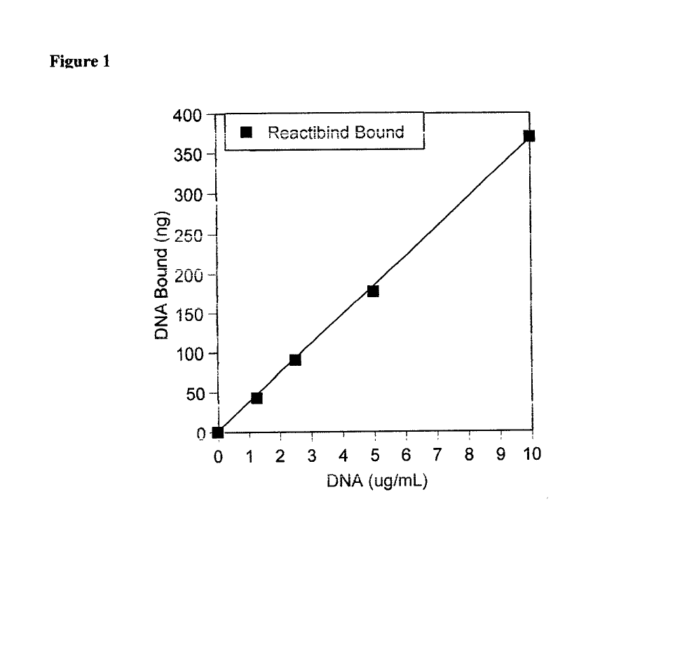

Next, to assure a sufficient and consistent quantity of DNA was bound to the plate, a commercially available, ultra-sensitive DNA quantitative fluorescent assay (PicoGreen) was performed following the manufacturer's instructions. FIG. 1 shows the amount of DNA bound to plate when DNA concentrations of 2.5, 5 and 10 μg/mL were incubated. About 100, 190, and 360 nanograms, respectively, of DNA were bound to each well.

Next, the open chain aldehyde in the DNA at AP sites was trapped by adding 100 μL of 1 mM of aldehyde-reactive probe (ARP) chemical reagent to each well of the plate, and the plate was incubated at room temperature for one hour. On the other hand, cells in culture may be incubated with ARP of equal concentration for one hour and the resulting DNA-ARP adduct in the cell can then be isolated and extracted by ethanol precipitation or an automated method.

Next, the excess ARP in each well was discarded from the plate, i.e., wells, were washed three to five times with a 1% Tween-PBS buffer (phosphate buffered solution containing 1% Tween 20) followed by washing once with 0.1% Tween-PBS. The plate was swiped and dried without desiccating. This reduced background noise, i.e., error, in the data from excess ARP to nil.

Next, 80 μL of 1:20 diluted ABC solution (horseradish peroxidase enzyme, which is commercially available from Vector Laboratories) was added to the wells having bound DNA. The plate was covered with parafilm and incubated at 37° C. for one hour and then washed with 0.1 % Tween-PBS buffer three to five times.

Next, 160 μL of 3,3,5,5-Tetramethylbenzidine (TMB) (commercially available from Moss Inc. of Pasadena, Md.) a substrate for horseradish peroxidase (other enzymes may be used for tagging biotin) was added to the wells and then the DNA was incubated at 37° C. (i.e., the optimum incubation temperature for enzyme being used where activity is highest) for 20-30 minutes until the reaction is completed. The absorbance was then measured at 650 nanometers wavelength using an optical bench plate reader.

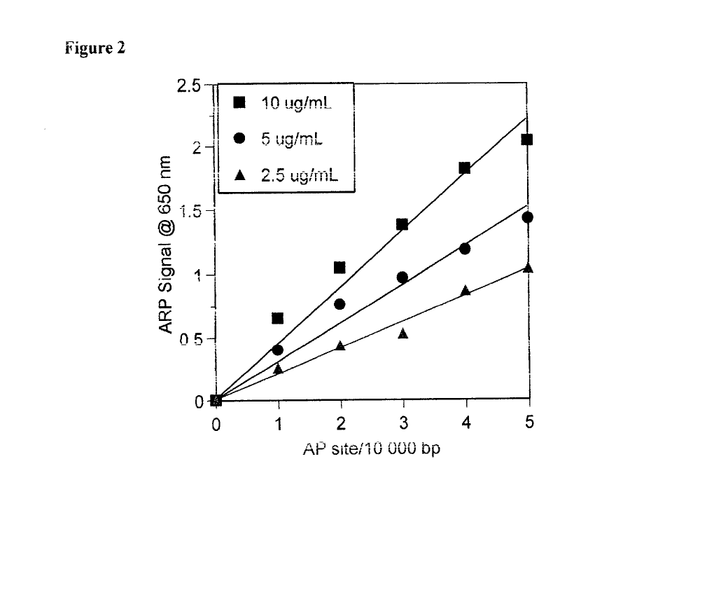

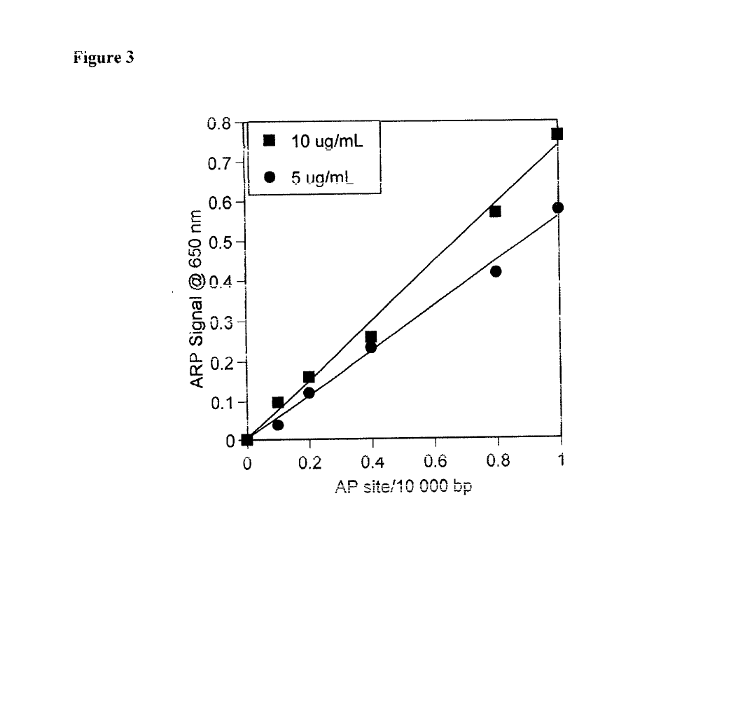

A standard or control against which to compare the sample DNA on the microtiter plate was created by selectively producing AP sites in DNA using heat/acid-buffer treatment of calf thymus (Lindahl & Nyberg, 1972, Kubo, et al., 1992). Relatively low-level AP sites in the DNA was produced at 0.1, 0.2, 0.4 and 0.8 AP site/10⁴ bp (base pairs), respectively. The resulting ARP signals are shown in FIGS. 2 and 3 respectively.

By varying the DNA concentrations and horseradish peroxidase substrate, the present invention provides assaying an abasic site quantity as low as 0.5 AP/10⁶bp of DNA.

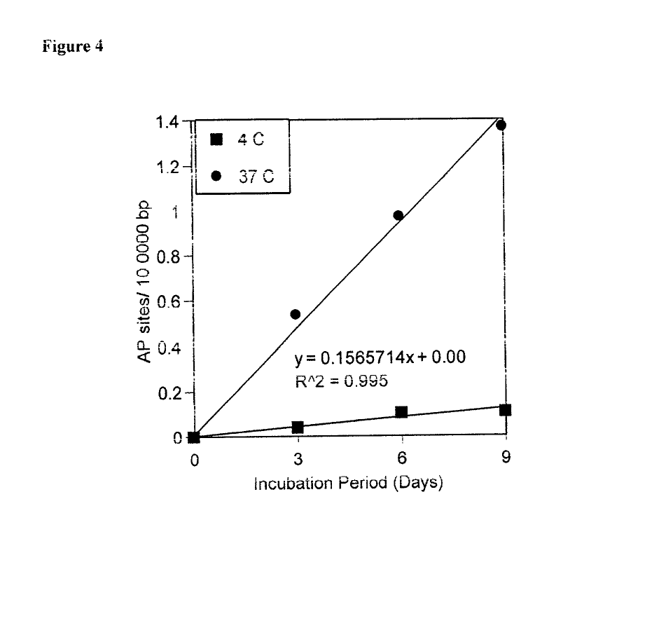

FIG. 4 shows the ARP signal as a measure of AP sites from spontaneous physiological depurination of calf thymus DNA, i.e. 37° C. and pH 7.0 for up to 10 days. The number abasic sites in the samples were then monitored with the present assay.

### EXAMPLE 2

Another procedure provides quantification of oxidative DNA base modification in DNA wherein the specific base modification includes N-glycosylase enzyme-sensitive modifications including thymine glycol, dihydrothymine, 8-hydroxyguanine and formamido remnant.

This procedure comprises contacting the damaged DNA with substrate specific N-glycosylase enzymes, such as endonuclease III or an fpg protein, ioslated and purified by an ether precipitation method resulting in the production of abasic (AP) site in DNA.

Using the method of Example 1 above, binding the genomic DNA to the microtiter plate. Aldehyde groups of the abasic site in the bound DNA are then labeled/tagged by an excess of a biotinylated aldehyde reactive chemical reagent (ARP).

Next, the method of Example 1 was used to prevent contributory nonspecific sites from excess reagent on the microtiter plate. The abasic (AP) sites contained in the tagged DNA were then quantitatively detected chromatographically using the microtiter plate by an avidin-biotin-horseradish peroxidase-conjugate method.

Next, an internal control DNA standard, similar to the samples, with specific inclusion of base modifications was similarly digested and the resulting number of abasic sites was determined as described above for accurate and sensitive quantitation.

Next, the quantity of abasic sites determined was found to be directly related to the quantity of specific N-gylcosylase sites (base modified substrate) in the damaged DNA.

Using the method of Example 1, when N-glycosylase (endonuclease) enzyme-coupling was used, it was possible to detect and quantify DNA base modifications including of 5-hydrox-5-methylhydantoin, 5-hydroxymethyluracil, or 8-hydroxyguanine and formamido reminants.

A 5,6-dihydroxyhydrothymine base modification (thymine glycol) was produced in calf thymus DNA in a dose dependent manner following exposure to osmium tetroxide by the procedure of Kow and Wallace (1985). The number of thymine glycols present in the DNA was previously determined (Hubbard et al., 1989). 0.01% OsO₄ treatment gives 1 fmol of thymine glycol, such that 0.04, 0.08 and 0.16% OsO₄ treatment gave 4, 8 and 16 fmol of thymine glycol respectively.

Next, each sample was completely processed using E.coli endonuclease III, isolated and purified as previously described at 5 uL/50 μg/mL DNA, and then reacted with ARP as described earlier.

In addition, the above method was applied to the resulting abasic sites after endonuclease III processing: tagged by the biotinylated ARP. The ARP assay was then used to determine the ARP signal in each sample.

The method of examples 1 and 2 can easily be provided in an assay kit to detect and quantify abasic (AP) sites in genomic a DNA sample isolated from cells and tissues.

### EXAMPLE 3

The invention further includes a method of determining DNA repair capacity of an isolated and purified biological (cells and tissues) homogenate that comprises comparing the optimum activity of the N-glycosylase enzyme in a purified biological homegenate of an individual with N-glycosylase enzyme activity of a commercial pure protein, and using the above-described quantitating methods to determine the DNA repair capacity of N-glycosylase enzyme in the isolated biological homogenate and the commercial N-glycosylase.

### EXAMPLE 4

The invention includes automation of the above-described methods to detect and quantitate abasic sites and oxidative DNA base modification in a sample of DNA using the ARP assay. Such methods are readily automated using conventional robotic techniques when coupled with automated methods of isolating DNA from cells, tissues and blood samples. The method of the present invention comprises (i) automated isolation of DNA from biological samples including cultured cells, tissue and blood presenting pure DNA; integration of the automated DNA isolation to direct microtiter plate, (ii) detection and quantitation of abasic sites and oxidative base modification in a sample of DNA as described in Example 1 using absorbance, and (iii) integration of the automated assay method to information systems for a fully automated and integrated method of detecting abasic sites and oxidative DNA base modification in a sample of DNA.

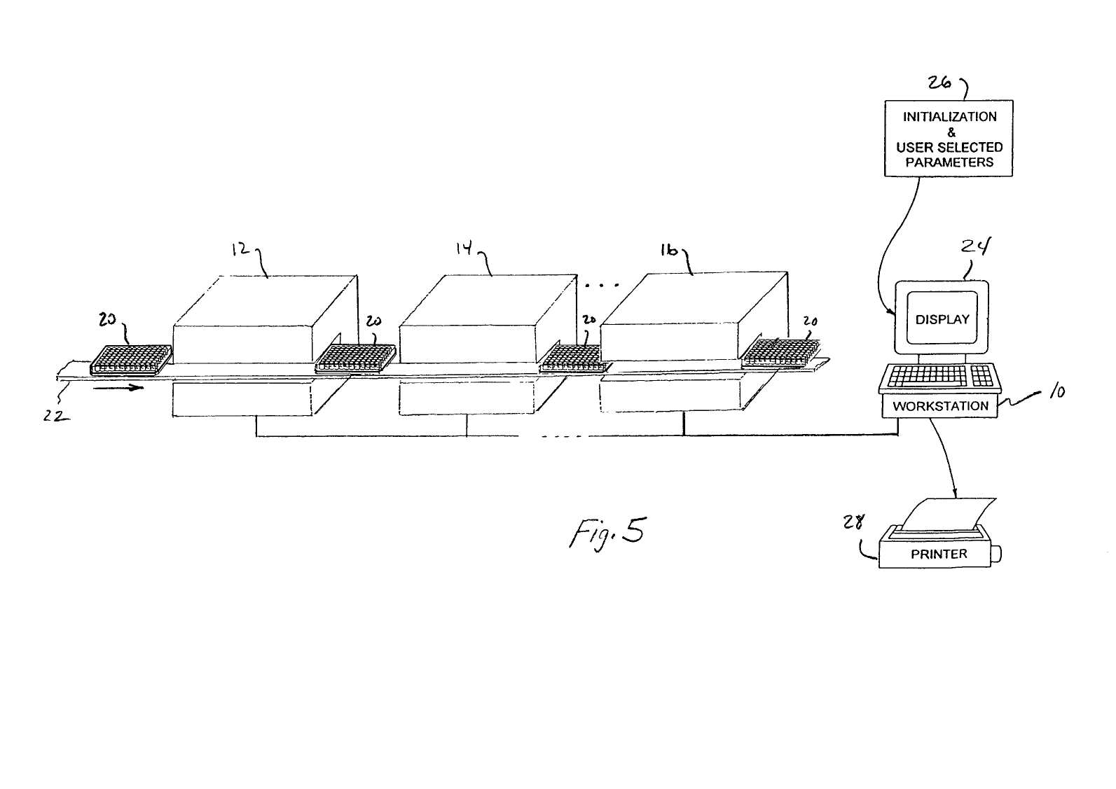

An exemplary apparatus that automatically performs DNA assays is conceptually illustrated in FIG. 5. Processor 10 implements software routines that control the functions of multiple processing stations 12, 14, and 16. As indicated above, the processing stations 12, 14, and 16, under control of processor 10, perform various functions relative to the microtiter plate 20 including dispensing control and/of sample DNA within respective wells of microtiter plate 20, applying binding reagents to the dispensed DNA to effect binding of DNA to the wells of plate 20, washing and drying the wells of plate 20 in a way so as to not remove bound DNA, applying an aldehyde reactive probe reagent to the DNA, controlling the environment within the processing station, performing optical density or absorbance measurements relative to DNA specimens in the respective wells.

The process descriptions contained herein also provide sufficient information as to how software routines can be developed to carry out the aforestated processes and functions using conventional numerical positioning controls and actuators, dispensers, environmental systems, drying/washing, etc. Processor 10 also effects movement of belt 22 in order to position the respective microtiter plates 20 at the respective processing stations and at the required times periods according methods described herein. Display 24 indicate the stages of processing of the DNA specimens and results of analyses. Parameters and conditions of processing for the respective samples may be provided by a memory 26. Printer 28 serves its customary function of printing results of DNA analyses.

### REFERENCES

Lindahl, T. and Nyberg, B. (1972) Biochemistry, 11, 3610-3618.

von Sonntag, C. (1987) Environ. Mol. Mutagen. 12, 431-477.

Rabow, J., Stubbe, J., Korazarich, J. W., and Gerlt, J. A. (1986) J. Am. Chem. Soc. 108, 7130-7131

Loeb, L. A., and Preston, B. D. (1986) Annu. Rev. Genet. 20, 201-230.

Wang, K. Y., Parker, S. A., Goljer, I. and Bolton, P. H. (1997) Biochemistry, 36, 11629-11639.

Sagher, D. and Strauss, B. (1983) Biochemistry, 22, 4518-4526.

Kamiya, H., Suzuki, M., Komotsu, Y., Miura, H., Kikuchi, K., Sakaguchi, T., Murata, N., Masutani, C., Hanaoka, F. and Ohtsuka, E. (1992) Nucleic Acids Res. 20, 4409-4415.

Kunkel, T. A. (1984) Proc. Natl. Acad. Sci. U.S.A. 81, 1494-1498.

Loeb, L. A., and Preston, B. D. (1986) Annu. Rev. Genet. 20, 201-230.

Mecoci, P., Polidori, M. C., Ingegni, T., Cherubini A., Chionne, F., Cecchetti R., Senin U (1998) Neurology 4, 1014-7.

Olkowski, Z. L. (1998) Neuroreport 9, 239-242

Mecocci, P. A., MacGarvey, U., and Beal, M. F. (1994) Ann Neurol 36, 747-51

Mecocci, P. A., Fano G., Fulle S., MacGarvey U., Shinobu, L., Polidori, M. C., Cherubini A., Vecchiet, J., Senin, U., Beal, M. F. (1999) Free Radic Biol Med, 3-4, 303-8

Lovell, M. A., Gabitta, S. P. and Markesbery, W. R. (1999) J. Neurochem., 72, 771-776.

Xu, Y., Moore, D. H., Broshears, J., Liu, L., Wilson, T. M., Kelley, M. R. (1997) Anticancer Res., 5B, 3713-19

Weinfeld, M., Liuzz, M., and Paterson, M. C. (1990) Biochemistry, 29, 1732-1743 Gilchrest et al., 1997

Kubo, K., Ide, H., wallace, S. S. and Kow, Y. W. (1992) Biochemistry, 31, 3703-8

Nakamura, J., Walker, V. E., Upton, P. B. S., Chiang Prashad et al., 1996

Baan, R. A., Van den Berg, P. T. M., Watson, W. P., nad Smith, R. J. (1988) In Bartsch, H., Kemminki, K. and O'Neil, I. K. (eds), Methods for Detecting DNA-Damaging Agents in Humans: Applications in Cancer Epidemiology and Prevention. IARC Scientific Publications, IARC, Lyon, No. 89, 146-154

Mori, T., Hori, Y., and Dizdaroglu, M. (1993) Inter J. Radiat. Biol, 64, 645-650 Bj.o slashed.r.ang.s, M., et al., “Opposite base-dependent reactions of a human base excision repair enzyme on DNA containing 7,8-dihydro-8-oxoguanine and abasic sites,” The EMBO Journal, 16:6314-6322 (1997).

Brow, M. D., et al., “Differentiation of Bacterial 16S rRNA Genes and Intergenic regions and Myobacterium tuberculosis katG Genes by Structure-Specific Endonulease Cleavage,” Journal of Clinical Microbiology, 34:3129-3137 (1996). Cannon, et al., “5-hydroxymethylcytosine DNA Glycosylase Activity in Mammalian Tissue,” Biochem, Biophys, Res. Commun. 151(3):1173-1179 (1988).

Castaing, B., et al. “Cleavage and binding of a DNA fragment containing a single 8-oxoguanine by wild type and mutant FPG proteins,” Nucleic Acids Research, 21:2899-2905 (1993).

Cotton, “Current Methods of Mutation Detection,” Mutation Research, 285:125-144 (1993).

Demple and Harrison, “Repair of Oxidative Damage to DNA,” Annu. Rev. Biochem., 63:915-48 (1994).

Devchand, P. R., et al., “Uracil-DNA glycosylase as a probe for protein-DNA interactions,” Nucleic Acids Research, 21:3437-3443 (1993).

Duncan, “DNA Glycosylases,” in The Enzymes, Boyer ed., p. 565-586 (1981).

Lindahl, “DNA Glycosylases, Endonucleases for Apurinic/Apyrimidinic Sites, and Base Excision-Repair,” Prog. Nucl. Acid Res. Mol. Biol. 22:135-192 (1979).

G. Hawkins and L. M. Hoffman, “Base excision sequence scanning, A new method for rapid sequences scanning and mutation detection,” Nature Biotechnology 15:803-804 (1997).

J. Tchou, et al., “8-Oxoguanine (8-hydroxyguanine) DNA glycosylase and its substrate specificity,” Procedure National Academy of Science USA, 88:4690-4694 (1991).

Maccubbin et al. “32-P Postlabeling assay for free radical-induced DNA damage:the formamido remnant of thymine”, Free Rad. Res. Comm. vol. 18, pp. 17-28, 1993.

Ide et al. “Thymine glycols and urea residues in M13 DNA constitute replicative blocks in vitro”, Nucleic Acids Research, vol. 13, pp. 8035-8052, 1985.

Maccubbin et al., P-Postlabeling Assay for Free Radical-Induced DNA Damage: The Formamido Remnant of Thymine, Sep. 13, 1992, “Free Rad. Res. Comms.”, vol. 18, No. 1, pp. 17-28.

Although the above embodiments and examples are described relative various reagents and enzymes, the invention is not limited to such. For example, Protamine Sulphate or other binding or immobilization techniques or solutions known in the art may be substituted for Reacti-bind. Other ELISA-like or calorimetric analysis techniques may be employed. Tagging or labeling may be accomplished with residues other than biotin. Other control DNA may be used beside calf thymus against which to compare the sample DNA. Washing may be performed using solutions other than buffered Tween 20 detergent. Comparing may be accomplished by other techniques, e.g., absorbance, optical density, color, etc., the object being to provide a basis for comparing sample and control DNA. Ordering of the steps described may be altered. Based on the foregoing description, one skilled in the art can easily ascertain modifications, substitutes, applications, and/or variations. It is the intent to include such modifications, substitutes, applications, and/or variations without departing from the spirit and scope of the invention.

What is claimed is:

1. A method of assaying DNA comprising:
(i) binding to an analysis plate both sample DNA under examination and control DNA having known abasic sites,
(ii) reacting the abasic sites with an aldehyde group-specific chemical reagent selected from a group of reagents including an aldehyde reactive probe (ARP) (N′-aminooxymethylcarbonylhydrazino-D-biotin) reagent whereby to attach the ARP to abasic sites,
(iii) using an ELISA-like method to detect abasic sites tagged with biotin after the reacting step wherein the ELISA-like method includes an avidin-biotin-complex conjugated with horseradish peroxidase or alkali phosphatase.

2. The method as recited in claim 1, comprising reacting ARP with AP sites of DNA of cells in culture before the binding step.
wherein the sample and control DNA are tagged or labeled separately with a the residue of the ARP reagent and then bound to the analysis plate for comparison.

3. The method as recited in claim 1, wherein the control DNA is a depurination of calf thymus.

4. The method as recited in claim 1, wherein the steps performed relative to the sample DNA and the control DNA are performed simultaneously so as to remove environmental or process variables at the comparing step.

5. The method as recited in claim 1, wherein Reacti-bind is used during the binding step.

6. The method as recited in claim 1, further including a washing step after the binding and labeling steps.

7. A method of quantitatively assaying DNA damage comprising the steps of binding to an analysis plate sample DNA and multiple control DNA specimens each having of known number of abasic sites, tagging aldehyde groups associated with abasic sites of the sample and control DNA, performing an ELISA-like method to obtain one of absorbance, optical density, and color density of the sample DNA and control DNA specimens, and comparing the sample DNA with multiple control DNA specimens to determine the number of abasic sites in the sample DNA.

8. A method of assaying repair capacity of sample DNA comprising:
treating sample and control DNA specimen with an enzyme that produces a substrate to which ARP attaches, reacting the sample and specimen DNA with ARP,
tagging aldehyde groups associated with abasic sites of the sample and control DNA,
performing an ELISA-like method to obtain at least one of absorbance, optical density, and color density of the sample DNA and control DNA specimens, and
comparing at least one of color, optical density, and absorbance of sample DNA with multiple control DNA specimens to determine relative enzyme activity levels of the sample and control DNA.

9. The method as recited in claim 8 wherein the treating step includes using an enzyme selected from the group including Endonuclease III, 8-oxoguanine glycosylase [yOGG1], human 8-oxoguanine glycosylase [hOGG1].

10. A method of assaying DNA comprising:
(i) binding sample and control DNA to an analysis plate to a microtiter plate using Reacti-bind wherein the control DNA has a known number of abasic sites,
(ii) removing the excess Reacti-bind and unbound DNA using a Tween 20 buffered detergent so as not to remove the bound DNA,
(iii) reacting the bound DNA with an excess amount of aldehyde reactive probe (ARP) reagent,
(iv) removing the excess and unreacted ARP from the analysis plate,
(v) labeling/tagging the attached ARP using a biotinylated chemical agent, and
(vi) performing a calorimetric analysis to quantitatively assess the sample DNA relative to the control DNA attached to the plate.

11. The method as recited in claim 10, further including binding to the analysis plate a relatively high percentage of DNA contained in a solution of relatively low concentration being in the range of 1.0 to 10.0 nanograms of DNA per milliliter.

12. A simplified kit for assaying sample DNA comprising:
an analysis plate, such as a microtiter plate;
a number of control DNA specimens of known concentration of abasic sites;
a surface treatment solution to enhance attachment of the DNA to the analysis plate;
a washing detergent for removing excess material from the plate;
an aldehyde reactive probe (ARP) to label or tag abasic sites while attached to the plate;
a colorimetric test kit such as a kit for performing an avidin-biotin horseradash peroxidase technique; and
instructions to provide a colorimetric comparison between sample DNA and control DNA by binding to an analysis plate sample DNA and multiple control DNA specimens each having of known number of abasic sites, tagging aldehyde groups associated with abasic sites of the sample and control DNA, performing an ELISA-like method to obtain at least one of absorbance, optical density, and color density of the sample DNA and control DNA specimens, and comparing the sample DNA with multiple control DNA specimens to determine the number of abasic sites in the sample DNA.

13. The simplified kit as recited in claim 12 further comprising a Reacti-bind solution for binding DNA to the analysis plate and an avidin biotin horseradish peroxidase enzyme for comparing enzyme digestion activity relative to the sample and control DNA.

14. A method for determining DNA repair capacity comprising the steps of:
subjecting sample and control DNA to a substrate specific repair enzyme,
tagging the product of the enzyme reaction,
binding the DNA to an analysis plate, and
determining the resulting number of abasic sites remaining on the analysis plate after the enzyme reaction whereby to assay the ability of the cell to undergo DNA repair.

15. The method as recited in claim 14, further comprising the steps of subjecting the sample and control DNA to a DNA glycosylase selected from the group of endonuclease III, N-glycosylase, 8-oxoguanine, alkA protein, and other broad and narrow spectrum DNA glycosylase.

16. An apparatus that automates assaying of DNA comprising:
an analysis plate to which sample and control DNA is bound,
a number of processing stations that perform process activities with respect to the analysis plate including at least one of binding DNA to a plate, reacting DNA with an aldehyde reactive probe reagent, tagging the ARP reagent, washing the DNA with a detergent, incubating the sample and control DNA, and
a controller that effect movement of the plate to the processing stations to perform the steps of binding to the analysis plate sample DNA and multiple control DNA specimens of known number of abasic sites, tagging aldehyde groups associated with abasic sites of the sample and control DNA, performing an ELISA-like method to obtain at least one of absorbance, optical density, and color of the sample DNA and control DNA specimens, and outputting an indication of a comparison of color density of the sample DNA relative to the control DNA specimens to indicate the number of abasic sites in the sample DNA.

17. The apparatus as recited in claim 16, wherein the controller effects operations of the processing stations to dispense sample and control DNA on the analysis plate; react a surface treatment agent including Reacti-bind with the sample and control DNA to bind them to the plate; wash the plate so as to remove excess surface treatment agent; reacting the bound DNA with an aldehyde reactive probe (ARP) so as to tag or label the DNA bound to the plate; biotinilating the ARP-tagged DNA; washing excess un-reacted ARP from the plate without removal of bound DNA; and outputting an indication of a calorimetric technique including an avidin-biotin horseradash peroxidase technique to indicate the number/concentration of abasic sites in the sample DNA.
